(19) United States
(12) Patent Application Publication
(10) Pub. No.: US 2024/0191310 A1
(43) Pub. Date: Jun. 13, 2024

(54) METHODS OF DETERMINING CANCER THERAPY EFFECTIVENESS

(71) Applicant: Strata Oncology, Inc., Ann Arbor, MI (US)

(72) Inventors: Daniel Reed Rhodes, Chelsea, MI (US); Scott Arthur Tomlins, Ann Arbor, MI (US); David Bryan Johnson, Chelsea, MI (US)

(21) Appl. No.: 18/571,660

(22) PCT Filed: Jun. 21, 2022
(86) PCT No.: PCT/US2022/034422

§ 371 Date: Dec. 18, 2023

Related U.S. Application Data
(60) Provisional application No. 63/212,581, filed on Jun. 18, 2021.

(51) Int. Cl. C12Q 1/6886; A61K 39/00; C07K 16/28; C07K 16/30; C07K 16/32

(52) U.S. Cl. CPC C12Q 1/6886; C07K 16/2803; C07K 16/30; C07K 16/32; A61K 2039/507; C12Q 2600/106; C12Q 2600/158

(57) ABSTRACT

Disclosed herein are methods of assessing whether a subject would be responsive to an immunotherapy.

### RELATED APPLICATION

This application claims priority to, and the benefit of, co-pending U.S. Provisional No. 63/212,581, filed Jun. 18, 2021. The disclosure of said provisional application is hereby incorporated by reference in its entirety.

### BACKGROUND OF THE INVENTION

Cancer immunotherapies are a new and effective form of treatment of cancers. For instance, checkpoint inhibitors (e.g., Anti-CTLA4/PD 1/PD-L1) have been widely used in cancer treatment and have impressive survival benefits. Additionally, other immunotherapies that utilize antibody drug conjugates, radiopharmaceuticals or bi-specific antibodies have also been brought to market which demonstrate effective targeting of activated oncogenes and CPIs. However, immunotherapies can cause a number of adverse events that can cause morbidity or mortality. For example, checkpoint inhibitors can cause colitis, hepatitis, adrenocorticotropic hormone insufficiency, hypothyroidism, type 1 diabetes, acute kidney injury or myocarditis. Antibody drug conjugates, radiopharmaceuticals and bi-specific antibodies are associated with one or more of grade 3 cytokine release syndrome, prolonged thrombocytopenia, ataxia, transaminitis, chemical pancreatitis, sino-occlusive syndrome, liver function abnormalities, neurotoxicity, angioedema, erythema multiform, renal failure, low oxygen perfusion, paresthesia, bradycardia and tachycardia, among others. Thus, it has become desirable to identify subjects with cancers responsive to these therapies prior to commencing treatment.

### SUMMARY OF THE INVENTION

In some aspects, the disclosure is directed to a method of determining if one or more therapies are likely to be effective for treating a subject, comprising measuring the expression level of one or more gene products associated with the one or more therapies from a biological sample obtained from the subject, wherein the sample comprises nucleic acids derived from cells targeted by the one or more therapies; measuring the expression level of at least one housekeeping gene selected from CIAO1, EIF2B1, and HMBS in the biological sample; normalizing the expression levels of the one or more gene products to the at least one housekeeping genes to obtain normalized expression levels of the one or more gene products; and determining that the one or more therapies are likely to be effective for treating the subject if the normalized expression levels of the one or more gene products are above a threshold level, wherein the therapy comprises an antibody, bispecific antibody, antibody-drug conjugate, antibody fragment, radiopharmaceutical, Car-T cell, or engineered T cell receptor (sometimes referred to herein as immunotherapies).

In certain embodiments, the one or more therapies comprise two or more therapies. In certain embodiments, the one or more therapies comprise three or more therapies.

In certain embodiments, the threshold level is the ranked expression level of the one or more gene products in a group of cells targeted by the one or more therapies corresponding to the percent of subjects not responsive to the one or more therapies.

In certain embodiments, the biological sample is a tumor sample.

In certain embodiments, the one or more therapies comprise an anti-Her2 antibody and the one or more gene products comprise Her2.

In certain embodiments, the one or more therapies comprise an anti-Nectin-4 antibody and the one or more gene products comprise Nectin-4.

In certain embodiments, the one or more therapies comprise an anti-TROP2 antibody and the one or more gene products comprise TROP2.

In certain embodiments, it is determined that at least two therapies are likely to be effective and wherein the therapy more likely to be effective is identified based on having the highest relative expression of the associated gene product over the associated threshold level.

In certain embodiments, the method further comprises determining if genomic DNA from the biological sample comprises one or more clinically relevant mutations.

In certain embodiments, it is determined if the one or more therapies or one or more mutation-directed targeted therapies would more likely be effective.

In certain embodiments, the method further comprises treating the subject with the one or more therapies or mutation-directed targeted therapy identified as likely to be effective.

In some aspects, the disclosure is directed to a method of determining if one or more antibody-based therapies selected from anti-Her2, anti-Nectin-4, and anti-TROP2 antibody-based therapy is likely to be effective for treating a tumor in a subject, comprising measuring the expression level of Her2, Nectin-4, and TROP2 in a tumor sample obtained from the subject; measuring the expression level of at least one housekeeping gene selected from CIAO1, EIF2B1, and HMBS in the tumor sample; normalizing the expression levels of Her2, Nectin-4, and TROP2 to the at least one housekeeping gene to obtain normalized expression levels of the one or more gene products; and determining that anti-Her2, anti-Nectin-4, or anti-TROP2 antibody-based therapy is likely to be effective if the normalized expression levels of Her2, Nectin-4, or TROP2 are above a threshold level.

In certain embodiments, the method further comprises determining which antibody-based therapy is more likely to be effective based on having the highest relative expression of the associated gene product over the associated threshold level.

In certain embodiments, further comprising determining if genomic DNA from the tumor sample comprises one or more clinically relevant mutations.

In certain embodiments, it is determined if the one or more antibody-based therapies or one or more mutation-directed targeted therapies would more likely be effective.

In some aspects, the disclosure is directed to a method of treatment of a subject with a therapy likely to be effective, comprising administering to said subject the therapy, wherein the therapy has been identified as likely to be effective by a method comprising measuring the expression level of a gene product associated with the therapy from a biological sample obtained from the subject, wherein the sample comprises nucleic acids derived from cells targeted by the therapy; measuring the expression level of at least one housekeeping gene selected from CIAO1, EIF2B1, or HMBS in the biological sample; normalizing the expression levels of the gene product to the at least one housekeeping gene to obtain a normalized expression level of the gene product; and determining that the therapy is likely to be effective for treating the subject if the normalized expression level of the gene product is above a threshold level, wherein the therapy comprises an antibody, bispecific antibody, antibody-drug conjugate, antibody fragment, radiopharmaceutical, Car-T cell, or engineered T cell receptor.

In certain embodiments, a plurality of therapies have been identified as likely to be effective and it has been determined that one of the therapies is more likely to be effective based on having the highest relative expression of the associated gene product over the associated threshold level; and the therapy identified as more likely to be effective is administered to the subject.

In some aspects, the disclosure is directed to a method of treatment of a subject with an antibody-based therapy identified as most likely to be effective selected from anti-Her2, anti-Nectin-4, and anti-TROP2 antibody-based therapy, comprising administering to said subject the antibody-based therapy identified as most likely to be effective, wherein the antibody-based therapy has been identified as most likely to be effective by a method comprising measuring the expression level of Her2, Nectin-4, and TROP2 in a tumor sample obtained from the subject; measuring the expression level of at least one housekeeping gene selected from CIAO1, EIF2B1, and HMBS in the tumor sample; normalizing the expression levels of Her2, Nectin-4, and TROP2 to the at least one housekeeping gene to obtain normalized expression levels of the one or more gene products; determining that anti-Her2, anti-Nectin-4, or anti-TROP2 antibody-based therapy is likely to be effective if the normalized expression levels of Her2, Nectin-4, or TROP2 are above a threshold level; and determining which antibody-based therapy is more likely to be effective based on having the highest relative expression of the associated gene product over the associated threshold level.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic depicting tumor to DNA and RNA co-isolation, sequencing, analysis and reporting and then treatment selection across targeted and antibody-directed therapies.

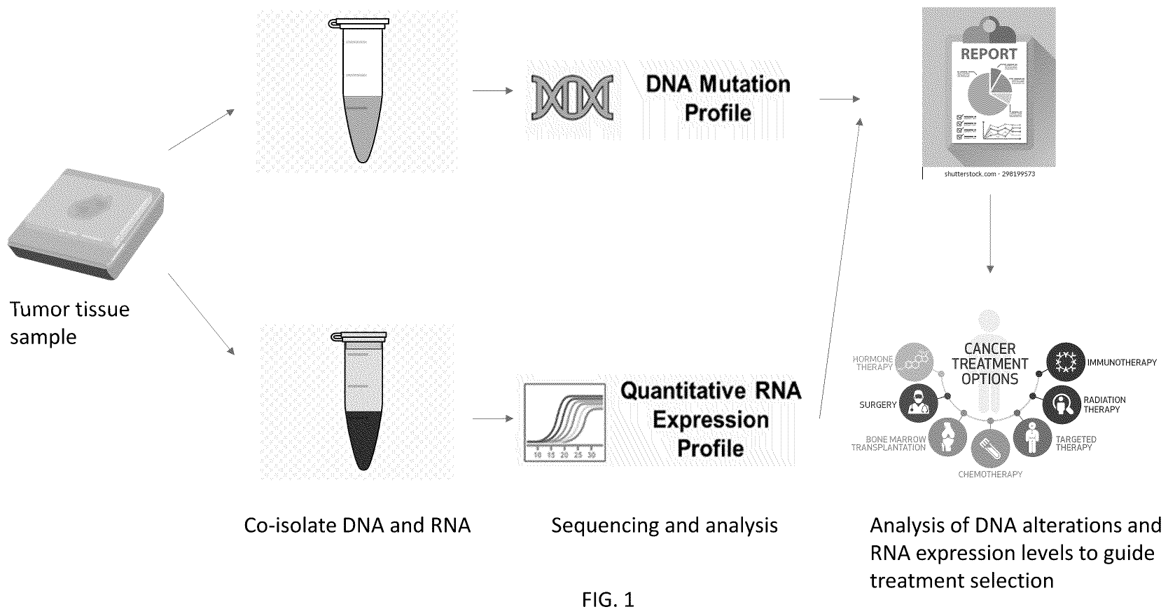

FIG. 2 depicts boxplots of the gene expression stability of candidate housekeeping genes across multiple RNA-seq panels. Genes EIF2B1, CIAO1 and HMBS had the most stable expression patterns in samples across multiple cancer types (n=29,533) by log 2 (normalized read per million (nRPM)) expression on (η=14.9, σ=0.30 for CIAO1; η=15.9, σ=0.42 for EIF2B1; n=13.0, o=0.62 for HMBS) and were thus chosen as the housekeeping genes for subsequent normalization of expression values. Furthermore, the standard deviation (s.d.) in the expression of the selected housekeeping genes is <4 s.d. away from the mean compared to other candidate housekeeping genes. Within each boxplot, the horizontal line represents the median, and the box boundaries below and above that represent subsequent quantile boundaries, with the whole distribution being subdivided evenly into the quantiles.

FIG. 3 is a plot which depicts the high concordance between gene expression determined by *StrataEXP℠ and qRT-PCR. 26 tissue samples were tested for 36 genes via StrataEXP℠ and qRT-PCR and demonstrated strong correlation with a square of the correlation coefficient r² being equal to 0.749.

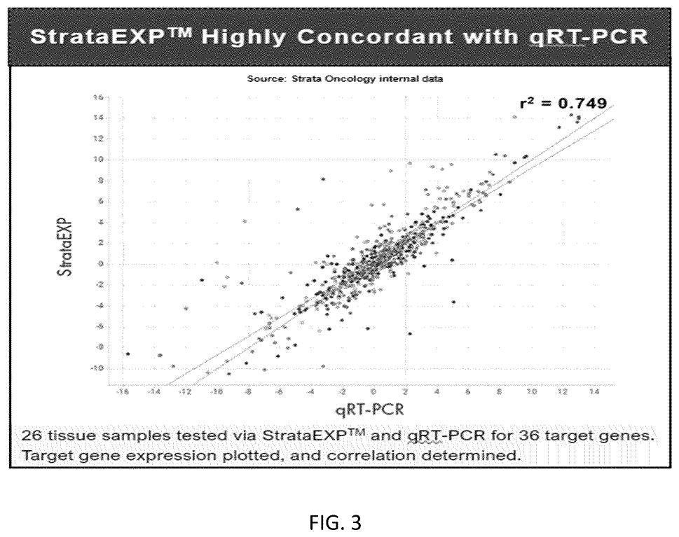

*StrataEXP℠ is described in more detail in paragraphs and [0139]-[0141] below.

FIGS. 4A-4D depict the StrataEXP℠ Nectin-4 threshold set by leveraging objective response rates to anti-Nectin-4 therapy in bladder cancer patients. FIGS. 4A-4B depict the objective response rates of 41% and 52% to the Nectin-4 directed antibody treatment enfortumab vedotin-ejfv in either a) a cohort of second line bladder cancer patients who received a prior PD-1 or PD-L1 inhibitor and were ineligible for cisplatin-containing chemotherapy or b) a cohort of third line bladder cancer patients who received a prior PD-L or PD-L1 inhibitor in addition to a platinum containing chemotherapy, respectively. FIG. 4C depicts the StrataEXP℠ Nectin-4 high/low expression threshold set by averaging the objective response rates to anti-nectin-4 immunotherapy in advanced or metastatic second or third line bladder cancer patients in recent clinical studies. FIG. D depicts box plots of the Nectin-4 solid tumor expression levels by tumor type, where the shading depicts 25th and 75th percentiles, respectively, and the black line depicts median expression. The red line depicts the StrataEXP℠ Nectin-4 high/low expression threshold of approximately 12.5 log 2 (nRPM). (¹ https://www.fda.gov/drugs/resources-information-approved-drugs/fda-grants-regular-approval-enfortumab-vedotin-ejfv-locally-advanced-or-metastatic-urothelial-cancer).

FIGS. 5A-5D depict the StrataEXP℠ TROP2 threshold set by leveraging objective response rates to anti-TROP2 therapy in bladder cancer patients. FIGS. 5A-5B depict the objective response rates of 34% and 39% to the TROP2 directed antibody treatment to Sacituzumab Govitecan-hziy in either a) a cohort of intention-to-treat second line bladder cancer patients or b) a cohort of response-evaluable second line bladder cancer patients. FIG. 5C depicts the StrataEXP℠ Nectin-4 high/low expression threshold set by averaging the objective response rates to anti-TROP2 immunotherapy in second line intent-to-treat or response-evaluable bladder cancer patients in recent clinical studies. FIG. 5D depicts box plots of the TROP2 solid tumor expression levels by tumor type, where the shading depicts 25th and 75th percentiles and the black line depicts median expression. The red line depicts the *StrataEXP℠ TROP2 high/low expression threshold of approximately 12.5 log 2 (normalized reads per million (nRPM)). (^{†}Intention-to-treat (ITT) patients include those who relapsed/progressed after CPI therapy; ^{‡}Response-evaluable (RE) patients received ≥2 doses and ≥ 1 post-baseline CT (RECIST 1.1) assessment, ¹. https://clinicaltrials.gov/ct2/show/NCT01631552; ². Tagawa S T, et al: Sacituzumab govitecan (IMMU-132) for patients with pretreated metastatic urothelial cancer (UC): interim results: 2017 ESMO. Abstract 3344. Annals of Oncology (2017) 28 (suppl_5): v295-v329.10.1093/annonc/mdx371)

FIGS. 6A-6C depict the StrataEXP℠ HER2 threshold set by leveraging the correlation between StrataEXP℠ determined HER2 expression with 3+ IHC HER2 expression in breast cancer samples as well as ROC curve to confirm appropriate threshold was selected. FIG. 6A depicts the correlation between HER2 expression levels in 312 breast cancer samples as determined by StrataEXP℠ and their correlation with 0, 1+, 2+ and 3+ HER2 expression status determined by immunohistochemistry. The selected StrataEXP℠ HER2 high/low expression threshold was approximately 19.3 log 2 (nRPM), which associated with a sensitivity of 77.3% and a specificity of 97.9%. FIG. 6B depicts a Receiver Operating Characteristic curve which confirms the threshold is set at an appropriate level. FIG. 6C depicts box plots of the HER2 solid tumor expression levels by tumor type, wherein n=23,514, and the red line depicts the StrataEXP℠ HER2 high/low expression threshold of approximately 19.3 log 2 (nRPM).

FIG. 7 depicts PD-L1 expression from 137 NSCLC patients as measured by StrataEXP℠ and stratified by orthogonally determined TPS score, showing higher expression in patients with TPS score of >=50% (green plot and points, n=45, η=13.1, σ=1.1) versus TPS <50% (blue plot and points, n=91, η=11.3, σ=2.3). Threshold for high expression is approximately 11.3 in log 2 (nRPM) and is delineated by the black dotted line, corresponding to 82.2% sensitivity and 82.4% specificity. TPS score is determined by dividing the number of PD-L1 stained tumor cells by the total number of viable tumor cells and multiplying by 100. A minimum of 100 viable tumor cells must be present in the PD-L1 stained slide for the specimen to be considered adequate for PD-L1 evaluation by the AGILENT 22C3/2808 pharmDX Assay. A tumor cell is counted as stained when any partial or complete membrane only staining of any intensity is identified. It is noted that while FFPE-tissues have been validated for use. Decalcified tissues or tissues processed with other fixatives (i.e., cytology specimens) have not been validated and are not recommended.

FIG. 8 displays StrataEXP℠ reported therapeutic RNA expression targets, with their associated therapies and example tumor type response rates.

### DETAILED DESCRIPTION OF THE INVENTION

Single-plex protein and genomic biomarkers on routine clinical formalin fixed paraffin embedded (FFPE) tissue specimens enabled initial precision oncology therapies such as those targeting lineage transcription factors (e.g., ER status by immunohistochemistry [IHC] in breast cancer), amplified tyrosine kinase receptors (e.g., ERBB2/HER2 amplification by fluorescent in situ hybridization [FISH] and HER2 over-expression by IHC in breast cancer), mutated tyrosine kinase receptors (e.g., Sanger sequencing or capillary electrophoresis of EGFR to detect single nucleotide variants [SNV] and short insertions/deletions [indels] in non-small cell lung cancer [NSCLC]), and tyrosine kinase gene fusions (e.g., FISH and reverse transcription PCR [RT-PCR] for ALK fusions in NSCLC).¹⁻⁶ The advent of massively parallel sequencing, also known as next generation sequencing (NGS), enabled translational research efforts to comprehensively define the cancer genome and transcriptome through whole exome and/or genome sequencing (WES/WGS) and whole transcriptome (RNAseq) sequencing of fresh frozen tissue specimens, such as The Cancer Genome Atlas (TCGA). Given cost, the limited number of targeted therapies, and required applicability to FFPE tissue specimens, initial clinical applications of NGS focused on small panels targeting oncogenic SNVs, indels, amplifications from DNA and gene fusions from RNA (using RT-PCR).

The parallel clinical development of therapies targeting tumor suppressors, such as PARP inhibitors in BRCA1 and BRCA2-deficient ovarian and prostate cancer, with decreasing NGS costs, enabled clinical NGS approaches targeting the complete coding sequence of hundreds of genes (often called comprehensive genomic profiling [CGP]), most notably the simultaneous FDA approval and CMS Medicare National Coverage Analysis Decision of FoundationOne™ CDx, a CGP test targeting the coding sequence of 327 genes from FFPE tumor DNA.

Simultaneously, the development of immunotherapy-based checkpoint inhibitors (CPIs) targeting CTLA4, PD-L1 (CD274) and its ligand PD-1 (PDCD1) revolutionized cancer therapy. Although some tumor types showed high response rates in unselected populations (e.g., melanoma and Merkel cell carcinoma), initial studies of pembrolizumab, a monoclonal antibody targeting PD-1, demonstrated that PD-L1 expression by IHC was predictive of response in NSCLC.⁷ Further clinical development has led to multiple complementary or companion diagnostic IHC assays based on PD-L1 expression to predict benefit of pembrolizumab and other PD-1/PD-L1 therapies in many tumor types.⁸⁻¹⁰ However, these assays vary based on staining platform, antibody clone, scoring methodology (e.g., expression in tumor cells, immune cells, or both) and cutoffs across tumor types, producing conflicting assessment of the “gold standard” PD-L1 protein expression by IHC even in the same sample, highlighting important limitations of current IHC target expression-based tests.⁸⁻¹²

Importantly, like other expression-based biomarkers not driven by high level amplifications (e.g., ER in breast cancer), PD-L1 cannot be measured by DNA based CGP. However, CPIs are thought to work in part by relieving the inhibition of T-cells that specifically recognize neoantigens created by somatic mutations in tumor cells, and early translational studies demonstrated that increasing tumor mutation burden (TMB; somatic mutations per megabase [Muts/Mb]) in individual tumor types was correlated with CPI response rate.¹³⁻¹⁸ In 2017, recognizing that microsatellite instable high (MSI-H)/mismatch repair deficient (dMMR) tumors (typically assessed by PCR-based MSI testing and MMR IHC) would have markedly increased neoantigen burden, Le et al. performed an investigator-initiated trial of pembrolizumab in patients with MSI-H/dMMR pan solid tumors.¹⁹ Combined with four other single arm studies totaling 149 patients with 15 types of MSI-H/dMMR tumors, these results led to the first tumor type-agnostic oncology approval by the FDA, with pembrolizumab approved for patients with any MSI-H/dMMR solid tumor based on an objective response rate of 40%, with 78% of responses lasting more than 6 months.²⁰ Critically, MSI-H can be directly assessed by CGP, representing an important complementary biomarker to standard PD-L1 IHC for predicting CPI benefit.

Most recently, after the explosion of therapies targeting activated oncogenes and CPIs, improved drug development approaches are enabling a third revolution in precision oncology for solid tumors with unprecedented development of expression-based therapies, such as antibody drug conjugates (ADCs), radiopharmaceuticals, and bi-specific antibodies. Since 2019, five such therapies have been FDA approved in advanced solid tumors, including trastuzumab deruxtecan targeting HER2 (ERBB2) in breast cancer, sacituzumab govitecan-hziy targeting TROP2 (TACTDS2) in urothelial carcinoma and triple-negative breast cancer, enfortumab vedotin targeting Nectin-4 (PVRL4) in urothelial carcinoma, tisotumab vedotin targeting Tissue Factor (F3) in cervical cancer, and Lutetium-177-PSMA-617 targeting PSMA (FOLH1) in prostate cancer. Critically, TROP2, Nectin-4, Tissue Factor and PSMA expression are not driven by underlying genomic alterations, and hence cannot be assessed by CGP; notably, although CGP can identify HER2 amplifications leading to 3+ IHC HER2 expression (the companion diagnostic biomarkers for trastuzumab deruxtecan in breast and gastric cancer), a phase III trial of trastuzumab deruxtecan in low HER2 expression (1-2+ IHC), which cannot be assessed by CGP, was recently reported as markedly increasing PFS and OS compared to chemotherapy²¹. Although sacituzumab, enfortumab and tisotumab do not have required biomarkers, target expression for sacituzuamb and enfortumab have recently been shown to be required for therapeutic efficacy²²⁻²⁴.

Prior to the advent of NGS, expression profiling predominantly by microarrays of fresh frozen tissues (nearly exclusively from surgical resection of non-metastatic tumors) largely defined the cancer transcriptome. Multiplex quantitative RT-PCR from FFPE cancer tissues (and subsequently microarray-based expression profiling) were clinically validated for therapy prediction and prognosis in early-stage cancers from several tumor types (e.g., gene expression profiling to guide adjuvant chemotherapy in ER+ breast cancer and refine prognosis in prostate cancer). However, such approaches do not assess genomic alterations and thus require subsequent CGP (or other genomic tests) if patients develop advanced disease.

Likewise, multiple studies, utilizing different RNA based expression approaches, have demonstrated that gene expression signatures, including those assessing both the tumor microenvironment (e.g. tumor infiltrating lymphocytes) and tumor expressed immune related markers such as CD8A and PD-L1, can better predict CPI response than PD-L1 IHC alone.²⁵⁻²⁹ Importantly, several of the same large, translational studies supporting TMB as predictive also demonstrated that such immune-related gene expression signatures were independent predictors of CPI benefit.^{26,30} Unfortunately, such integrative biomarkers have largely been developed on research platforms or clinical platforms that require additional CGP platforms for genomic alteration assessment (e.g., TMB by whole exome sequencing and immune gene expression by Nanostring).²⁶

Lastly, although capture-based transcriptome sequencing, which could enable individual biomarker and multigene/integrative DNA and RNA signature assessment, has been reported to be robust from FFPE tissue, formal validation of the quantitative nature of such approaches has not been reported. For example, although Shohdy et al. demonstrated that three different capture transcriptome sequencing approaches on FFPE tissue showed high rank correlation (Spearman r ˜0.6-0.8) across the transcriptome vs. parallel fresh frozen tissues subjected to whole transcriptome, no quantitative correlation was presented³¹ and the gold standard for gene expression quantification is qRT-PCR, as other studies have shown that multiplex PCR based NGS has ˜1000× greater dynamic range vs. whole transcriptome sequencing in high quality fresh frozen RNA31. Hence, the need remains for analytically validated, clinically relevant transcriptional profiling to complement CGP, which often must be performed in challenging FFPE tissue samples.

We previously reported the analytical validation and clinical performance (across >30,000 consecutively submitted FFPE tumor samples) of StrataNGS®, a comprehensive genomic profiling (CGP) test^{32,33}. StrataNGS® begins with co-isolation of DNA and RNA, as RNA is subjected to separate RT-PCR and a multiplex PCR library preparation with over 950 individual gene fusion isoforms involving 59 targeted driver gene fusions. In parallel, amplicons targeting clinically relevant non-chimeric expression targets have been included in the multiplex library preparation to potentially enable integrative DNA and RNA based predictive signatures, multi-gene expression signatures, and individual expression biomarker quantification to enable improved precision oncology beyond CGP alone.

StrataEXP℠ performs quantitative gene expression assessment by targeted, multiplex RNAseq from the same RNA used to assess gene fusions in StrataNGS®. StrataEXP℠ can be used to identify patients that would benefit from FDA approved medications (i.e., antibody drug conjugates) or investigational therapies, and may be used for clinical trial enrollment consideration requiring biomarker assessment.

An advantage of StrataEXP℠ is in respect to clinical utility on small routine formalin fixed, paraffin embedded (FFPE) tumor tissue samples (≥2 mm2 tumor surface area). This low tissue requirement allows up to 50% more samples to be tested than most currently commercially available CGP tests.³³

### Some Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “antibody” herein is used in the broadest sense and encompasses various antibody structures, including but not limited to monoclonal antibodies, polyclonal antibodies, and multispecific antibodies (e.g., bispecific antibodies).

The term “monoclonal antibody” as used herein refers to an antibody obtained from a population of substantially homogeneous antibodies, i.e., the individual antibodies comprising the population are identical and/or bind the same epitope. except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “human antibody” is an antibody that possesses an amino acid sequence corresponding to that of an antibody produced by a human and/or has been made using any of the techniques for making human antibodies known to one of skill in the art. This definition of a human antibody specifically excludes a humanized antibody comprising non-human antigen-binding residues. Human antibodies can be produced using various techniques known in the art, including methods described in Cole et al. Monoclonal Antibodies and Cancer Therapy, Alan R. Liss, p. 77 (1985); Boerner et al, J. Immunol, 147(I):86-95 (1991). See also van Dijk and van de Winkel, Curr. Opin. Pharmacol, 5: 368-74 (2001). Human antibodies can be prepared by administering the antigen to a transgenic animal that has been modified to produce such antibodies in response to antigenic challenge, but whose endogenous loci have been disabled, e.g., immunized HuMab mice (see, e.g., Nils Lonberg et al., 1994, Nature 368:856-859, WO 98/24884, WO 94/25585, WO 93/1227, WO 92/22645, WO 92/03918 and WO 01/09187 regarding HuMab mice), xenomice (see, e.g., U.S. Pat. Nos. 6,075,181 and 6,150,584 regarding XENOMOUSE™ technology) or Trianni mice (see, e.g., WO 2013/063391, WO 2017/035252 and WO 2017/136734 regarding Trianni mice).

The term “humanized antibody” refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those skilled in the art will be aware of humanized antibodies and will also be aware of suitable techniques for their generation. See for example, Hwang. W. Y. K., et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27.1988; Verhoeyen et al., Science, 239:1534-36, 1988; Orlandi et al., Proc. Natl. Acad. Sci. USA. 86:3833-37, 1989; U.S. Pat. Nos. 5,225,539; 5,530,101; 5,585,089; 5,693,761; 5,693,762; 6,180,370; and Selick et al., WO 90/07861, each of which is incorporated herein by reference in its entirety.

As used herein, the term “bispecific antibodies” refers to monoclonal, often human or humanized, antibodies that have binding specificities for at least two different antigens. In the invention, one of the binding specificities can be directed towards CLDN18.2, the other can be for any other antigen, e.g., for a cell-surface protein, receptor, receptor subunit, tissue-specific antigen, virally derived protein, virally encoded envelope protein, bacterially derived protein, or bacterial surface protein, etc.

The term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab)₂; diabodies; linear antibodies; single-chain antibody molecules (e.g., scFv). Papain digestion of antibodies produces two identical antigen-binding fragments, called “Fab” fragments, and a residual “Fc” fragment, a designation reflecting the ability to crystallize readily. The Fab fragment consists of an entire light (L) chain (VL) along with the variable region domain of the heavy (H) chain (VH), and the first constant domain of one heavy chain (CH1). Pepsin treatment of an antibody yields a single large F(ab)₂ fragment which roughly corresponds to two disulfide linked Fab fragments having divalent antigen-binding activity and is still capable of cross-linking antigen. Fab fragments differ from F(ab)₂ fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “expression” refers to the cellular processes involved in producing RNA and proteins and as appropriate, secreting proteins, including where applicable, but not limited to, transcription, translation, folding, modification and processing. “Expression products” include RNA transcribed from a gene and polypeptides obtained by translation of mRNA transcribed from a gene.

The term “RNA” is defined as ribonucleic acid.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The terms “non-human animals” and “non-human mammals” as used herein interchangeably, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

### Methods of Determining Effective Therapies

In some aspects, the disclosure is directed to a method of determining if one or more therapies (i.e., immunotherapies) are likely to be effective for treating a subject, comprising measuring the expression level of one or more gene products associated with the one or more therapies from a biological sample obtained from the subject, wherein the sample comprises nucleic acids derived from cells targeted by the one or more therapies; measuring the expression level of at least one housekeeping gene selected from CIAO1, EIF2B1, and HMBS in the biological sample; normalizing the expression levels of the one or more gene products to the at least one housekeeping gene to obtain normalized expression levels of the one or more gene products; and determining that the one or more therapies are likely to be effective for treating the subject if the normalized expression levels of the one or more gene products are above a threshold level, wherein the therapy comprises an immunotherapy (e.g., comprising an antibody, bispecific antibody, antibody-drug conjugate, antibody fragment, radiopharmaceutical, Car-T cell, or engineered T cell receptor).

In some embodiments, the expression level of at least two housekeeping genes selected from CIAO1, EIF2B1, and HMBS are measured. In some embodiments, the expression level of all three housekeeping genes selected from CIAO1, EIF2B1, and HMBS are measured.

The cancer immunotherapies assessed by the methods disclosed herein are not limited and may be any suitable immunotherapy having a gene product associated with the immunotherapy. In some embodiments, the immunotherapy targets a growth factor or growth factor receptor (e.g., EGFR, EGFRVIII, HER2, HER3, PDGF, PDGFR, HGF, HGFR, IGF, IGFIR, VEGF, VEGFR, TGFb, TGFbR, FGF, or FGFR). In some embodiments, the immunotherapy targets a tumor cell surface molecule (e.g., CA125, CA19-9, CD30, CEACAM5, CEACAM1, CEACAM6, DLL3, DLL4, DPEP3, EGFR, EGFRVIII, GD2, HER2, HER3, HGF, IGFIR, IL13Ra2, LIV-1, LRRC15, MUC1, PRLR, PSCA, PSMA, PTK7, SEZ6, SLAMF7, TF, cMet, claudin, mesothelin, nectin4, uPAR, GPNMB, CD79b, CD22, NaPi2b, SLTRK6, STEAP1, MUC16, CD37, GCC. AGC-16, 5T4, CD70, TROP2, CD74, CD27L, Fra, CD138, or CA6). In some embodiments, the immunotherapy comprises an antibody selected from MEDI2228; CC-99712; belantamab; Gemtuzumab (anti-CD33 mAb), rituximab (chimeric murine/human anti-CD20 mAb); Obinutuzumab (anti-CD20 mAb); Ofatumumab (anti-CD20 mAb); Tositumumab-1131 (anti-CD20 mAb); Ibritumomab tiuxetan (anti-CD20 mAb). In some embodiments, the immunotherapy comprises an obligate or non-obligate bsAb. In some embodiments, one of the targets of the bsAb is CD3. In one aspect, the bsAb may be a CrossMab or a BiTE. Examples of bsAbs that may be used as targeting polypeptides of the fusion proteins of the invention include the following: CD3×B7-H3 (e.g., orlotamab), CD3×BCMA (e.g., AMG420, AMG701, EM801, JNJ-64007957, PF-06863135, REGN5458), CD3×CD19 (e.g., A-319, AFM11, AMG562, blinatumomab), CD3×CD20 (e.g., mosunetuzumab, plamatomab, REGN1979, CD20-TCB), CD3×CD33 (e.g., AMG330, AMG673, AMV-564, GEM333), CD3×CD38 (e.g., AMG424, GBR1342), CD3×CEA (e.g., Cibisatamab), CD3×EGFRvIII (e.g., AMG596), CD3×EpCAM (e.g., A-337, catumaxomab, removab), CD3×FLT3 (e.g., AMG427), CD3×GPC3 (e.g., ERY974), CD3×gpA33 (e.g., MGD007), CD3×GPRC5D (e.g., JNJ-64407564), CD3×HER2 (e.g., GBR1302, M802, RG6194), CD3×MUC16 (e.g., REGN4018), CD3×P-Cadherin (e.g., PF-06671008), CD3×PSMA (e.g., AMG160, MOR209, pasotuxizumab), CD3×SSTR2 (e.g., tidutamab), CD40×MSLN (e.g., ABBV-428), PD-1×ICOS (e.g., Xmab23104), or PD-L1×4-1BB (e.g., MCLA-145), or CTLA-4×PD-1 In some embodiments, the immunotherapy targets CD47, SIRPa, CD31, CD24, SIGLEC10, or LILRB1. In some embodiments, the immunotherapy targets a T cell inhibitory receptor (TCIR), a T cell inhibitory receptor ligand (TCIR ligand), a T-cell co-inhibitory molecule, or a T cell co-stimulatory molecule. In some embodiments, the immunotherapy targets Cytotoxic T lymphocyte associated antigen-4 (CTLA-4, CD152), Programmed Death-1 protein (PD-1), Programmed death ligand-1 (PD-L1), Programmed death ligand (PD-L2), B7-H3 (CD276), T-cell immunoglobulin and mucin-domain containing-3 (TIM-3), Carcinoembryonic antigen-related cell adhesion molecule (CEACAM), V domain Ig suppressor of T cell activation (VISTA), V-set and immunoglobulin domain containing 8 (VSIG8), B and T lymphocyte attenuator (BTLA), Herpesvirus Entry Mediator (HVEM), CD160, T cell Ig and ITIM domain (TIGIT), PVRIG, CD226, CD96, Lymphocyte activation gene-3 (LAG-3). In some embodiments, the immunotherapy targets 4-1BB (CD137), Inducible T-Cell Costimulator (ICOS), OX-40 (CD134), Herpesvirus Entry Mediator (HVEM), glucocorticoid-induced TNFR-related protein (GITR), CD40, CD30, DNAM, or CD27. In some embodiments, the immunotherapy targets IL-17, IL-17R, IL-23, IL-23R, IL-6, IL-6R, IL-1, IL-1R, IL-10, IL-10R, TGFb, or TGFbR.

In some embodiments, the therapy targets TROP2; PD-L1; Nectin-4; HER2; LIV-1; NaPi2b; FOLR1; PSMA; or androgen receptor (AR).

In some embodiments, the immunotherapies target HER2. In some embodiments, the immunotherapy comprises at least one selected from the group consisting of trastuzumab, trastuzumab-emtansine, tucatinib, pyrotinib, pertuzumab, and trastuzumab-deruxtecan. In some embodiments, the immunotherapy comprises trastuzumab. In some other embodiments, the immunotherapies target PD-1/PD-L1 across tumor types. In some embodiments the immunotherapy comprises at least one of pembrolizumab, nivolumab, cemiplimab-rwlc and atezolizumab. In some embodiments the immunotherapies target PIK3CA mutations in ER⁺/HER2⁻ breast cancer. In some embodiments, the immunotherapy comprises alpelisib. In some embodiments, the immunotherapy targets Nectin-4 overexpression. In some embodiments, the immunotherapy targets Nectin-4 overexpression in metastatic urothelial cancer. In some embodiments the immunotherapy comprises enfortumab vedotin. In some embodiments, the immunotherapy targets TROP2 overexpression. In some embodiments the immunotherapy comprises Sacituzumab. In some further embodiments, the immunotherapy comprises both Sacituzumab and enfortumab vedotin and targets both TROP2 overexpression and Nectin-4 overexpression in metastatic urothelial cancer.

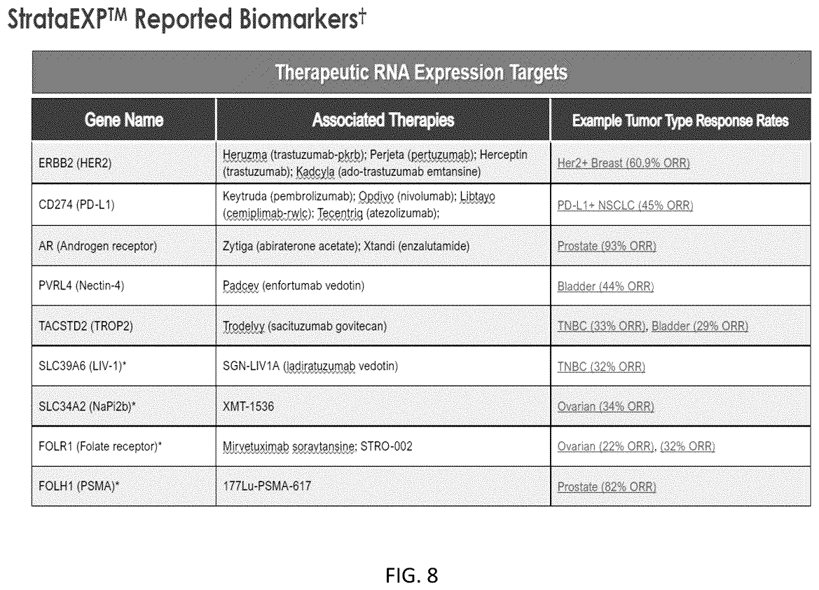

In some embodiments, the therapy determined to likely be effective in treating a subject, is at least one of the therapies outlined in FIG. 8.

In certain embodiments, the one or more therapies comprise two or more therapies. In certain embodiments, the one or more therapies comprise three or more therapies.

In certain embodiments, the threshold level is the ranked expression level of the one or more gene products in a group of cells targeted by the one or more therapies corresponding to the percent of subjects not responsive to the one or more therapies.

In some embodiments, the threshold for selected immunotherapies associated with TROP2; PD-L1; Nectin-4; HER2; LIV-1; NaPi2b; FOLR1; PSMA; or androgen receptor (AR) are within the following range (all log 2 (normalized reads per million) after centering so the overall population median is 10):
TROP2: 12-13
PD-L1: 11-12
Nectin-4: 12-13
HER2: 18-20
LIV-1: 11-12
NaPi2b: 17-18
FOLR1: 16.5-17.5
PSMA: 13.5-14.5
AR: 14-15

In some embodiments, the threshold for selected immunotherapies associated with TROP2; PD-L1; Nectin-4; HER2; LIV-1; NaPi2b; FOLR1; PSMA; or androgen receptor (AR) are approximately as follows (all log 2 (normalized reads per million) after centering so the overall population median is 10):

TROP2: 12.5
PD-L1: 11.3
Nectin-4: 12.5
HER2: 19.3
LIV-1: 11.6
NaPi2b: 17.5
FOLR1: 17.0
PSMA: 14.0
AR: 14.4

In certain embodiments, the one or more therapies comprise an anti-Her2 antibody and the one or more gene products comprise Her2. In certain embodiments, the one or more therapies comprise an anti-Nectin-4 antibody and the one or more gene products comprise Nectin-4. In certain embodiments, the one or more therapies comprise an anti-TROP2 antibody and the one or more gene products comprise TROP2.

In embodiments of the methods disclosed herein, the biological sample (i.e., sample) is any suitable sample type. In some embodiments, the sample is from plasma, blood, serum, saliva, sputum, stool, a tumor, cell free DNA, circulating tumor cell, or other biological sample. In some embodiments, the sample is a blood sample. In some embodiments, the biological sample is a tumor specimen. In some embodiments, the sample is from a subject having or at risk of having cancer. The type of cancer is not limited and may be any suitable cancer. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is lung or prostate cancer.

In some embodiments, the cancer is selected from adrenal, biliary, bladder, brain, breast, cervical, colon and rectum, endometrium, esophagus, head and neck, kidney, liver, lung—NSCLC, lung—Other, lymphoma, melanoma, meninges, NSCLC, non-melanoma skin, ovary, pancreas, prostate, sarcoma, small intestine, stomach, thymus, or thyroid cancer. In some embodiments, the cancer is selected from lung, bladder, colon, gastric, head and neck, breast, prostate, non-small cell lung adenocarcinoma, non-small cell lung squamous cell carcinoma, bladder urothelial carcinoma, colorectal, brain or pancreatic cancer.

In certain embodiments, it is determined that at least two therapies are likely to be effective and wherein the therapy more likely to be effective is identified based on having the highest relative expression of the associated gene product over the associated threshold level. In some embodiments, the therapy more likely to be effective has at least a 5%, 10%, 25%, or greater relative expression over the associated threshold level than the other therapy.

In certain embodiments, the method further comprises determining if genomic DNA from the biological sample comprises one or more clinically relevant mutations. In some embodiments, the clinically relevant mutation is a BRAF, KIT, NF, NRAS, or PTEN mutation.

In certain embodiments, it is determined if the one or more therapies or one or more mutation-directed targeted therapies would more likely be effective. In certain embodiments, the method further comprises treating the subject with the one or more therapies or mutation-directed targeted therapy identified as likely to be effective.

In some aspects, the disclosure is directed to a method of determining if one or more antibody-based therapies selected from anti-Her2, anti-Nectin-4, and anti-TROP2 antibody-based therapy is likely to be effective for treating a tumor is a subject, comprising measuring the expression level of Her2, Nectin-4, and TROP2 in a tumor sample obtained from the subject; measuring the expression level of at least one housekeeping gene selected from CIAO1, EIF2B1, and HMBS in the tumor sample; normalizing the expression levels of Her2, Nectin-4, and TROP2 to the at least one housekeeping gene to obtain normalized expression levels of the one or more gene products; and determining that anti-Her2, anti-Nectin-4, or anti-TROP2 antibody-based therapy is likely to be effective if the normalized expression levels of Her2, Nectin-4, or TROP2 are above a threshold level.

As used herein, an “antibody-based” therapy is an immunotherapy with an antibody to a therapeutic target. Usually, said antibody-based therapy comprise an antibody conjugated to a toxic (e.g., an antibody-drug conjugate). These immunotherapies are not limited and may be any suitable immunotherapy targeting Her2, Nectin-4, or TROP2.

In some embodiments, the therapy targeting Her2 is trastuzumab, pertuzumab, margetuximab, Ado-trastuzumab emtansine, and/or fam-trastuzumab deruxtecan. In some embodiments, the therapy targeting Nectin-4 is enfortumab vedotin. In some embodiments, the therapy targeting TROP2 is Sacituzumab.

In some embodiments, the expression level of at least two housekeeping genes selected from CIAO1, EIF2B1, and HMBS are measured. In some embodiments, the expression level of all three housekeeping genes selected from CIAO1, EIF2B1, and HMBS are measured.

In certain embodiments, the method further comprises determining which antibody-based therapy is more likely to be effective based on having the highest relative expression of the associated gene product over the associated threshold level. In some embodiments, the therapy more likely to be effective has at least a 5%, 10%, 25%, or greater relative expression over the associated threshold level than the other therapy.

In some embodiments, the threshold for selected immunotherapies associated with TROP2; Nectin-4; or HER2 are within the following range (all log 2 (normalized reads per million) after centering so the overall population median is 10):
TROP2: 12-13
Nectin-4: 12-13
HER2: 18-20

In some embodiments, the threshold for selected immunotherapies associated with TROP2; Nectin-4; or HER2 are approximately as follows (all log 2 (normalized reads per million) after centering so the overall population median is 10):
TROP2: 12.5
Nectin-4: 12.5
HER2: 19.3

In certain embodiments, further comprising determining if genomic DNA from the tumor sample comprises one or more clinically relevant mutations. In some embodiments, the clinically relevant mutation is a BRAF, KIT, NF, NRAS, or PTEN mutation.

In certain embodiments, it is determined if the one or more antibody-based therapies or one or more mutation-directed targeted therapies would more likely be effective.

### Methods of Treatment

In some aspects, the disclosure is directed to a method of treatment of a subject with a therapy (i.e., immunotherapy) likely to be effective, comprising administering to said subject the therapy, wherein the therapy has been identified as likely to be effective by a method comprising measuring the expression level of a gene products associated with the therapy from a biological sample obtained from the subject, wherein the sample comprises nucleic acids derived from cells targeted by the therapy; measuring the expression level of at least one housekeeping gene selected from CIAO1, EIF2B1, and HMBS in the biological sample; normalizing the expression levels of the gene product to the at least one housekeeping gene to obtain a normalized expression level of the gene product; and determining that the therapy is likely to be effective for treating the subject if the normalized expression level of the gene product is above a threshold level, wherein the therapy comprises an immunotherapy (e.g., comprising an antibody, bispecific antibody, antibody-drug conjugate, antibody fragment, radiopharmaceutical, Car-T cell, or engineered T cell receptor).

In some embodiments, the expression level of at least two housekeeping genes selected from CIAO1, EIF2B1, and HMBS are measured. In some embodiments, the expression level of all three housekeeping genes selected from CIAO1, EIF2B1, and HMBS are measured.

The therapies are not limited and may be any therapy (immunotherapy) described herein. In some embodiments, the therapy is any suitable immunotherapy having a gene product associated with the immunotherapy.

In certain embodiments, a plurality of therapies have been identified as likely to be effective and it has been determined that one of the therapies is more likely to be effective based on having the highest relative expression of the associated gene product over the associated threshold level; and the therapy identified as more likely to be effective is administered to the subject. In some embodiments, the therapy more likely to be effective has at least a 5%, 10%, 25%, or greater relative expression over the associated threshold level than the other therapy.

In some aspects, the disclosure is directed to a method of treatment of a subject with an antibody-based therapy identified as most likely to be effective selected from anti-Her2, anti-Nectin-4, and anti-TROP2 antibody-based therapy, comprising administering to said subject the antibody-based therapy identified as most likely to be effective, wherein the antibody-based therapy has been identified as most likely to be effective by a method comprising measuring the expression level of Her2, Nectin-4, and TROP2 in a tumor sample obtained from the subject; measuring the expression level of at least one housekeeping gene selected from CIAO1, EIF2B1, and HMBS in the tumor sample; normalizing the expression levels of Her2, Nectin-4, and TROP2 to the at least one housekeeping gene to obtain normalized expression levels of the one or more gene products; determining that anti-Her2, anti-Nectin-4, or anti-TROP2 antibody-based therapy is likely to be effective if the normalized expression levels of Her2, Nectin-4, or TROP2 are above a threshold level; and determining which antibody-based therapy is more likely to be effective based on having the highest relative expression of the associated gene product over the associated threshold level.

As used herein, an “antibody-based” therapy is an immunotherapy with an antibody to a therapeutic target. Usually, said antibody-based therapy comprise an antibody conjugated to a toxic (e.g., an antibody-drug conjugate). These immunotherapies are not limited and may be any suitable immunotherapy targeting Her2, Nectin-4, or TROP2.

In some embodiments, the therapy targeting Her2 is trastuzumab, pertuzumab, margetuximab, Ado-trastuzumab emtansine, and/or fam-trastuzumab deruxtecan. In some embodiments, the therapy targeting Nectin-4 is enfortumab vedotin. In some embodiments, the therapy targeting TROP2 is Sacituzumab.

In some embodiments, the expression level of at least two housekeeping genes selected from CIAO1, EIF2B1, and HMBS are measured. In some embodiments, the expression level of all three housekeeping genes selected from CIAO1, EIF2B1, and HMBS are measured.

In certain embodiments, the method further comprises determining which antibody-based therapy is more likely to be effective based on having the highest relative expression of the associated gene product over the associated threshold level. In some embodiments, the therapy more likely to be effective has at least a 5%, 10%, 25%, or greater relative expression over the associated threshold level than the other therapy.

In some embodiments, the threshold for selected immunotherapies associated with TROP2; Nectin-4; or HER2 are within the following range (all log 2 (normalized reads per million) after centering so the overall population median is 10):
TROP2: 12-13
Nectin-4: 12-13
HER2: 18-20

In some embodiments, the threshold for selected immunotherapies associated with TROP2; Nectin-4; or HER2 are approximately as follows (all log 2 (normalized reads per million) after centering so the overall population median is 10):
TROP2: 12.5
Nectin-4: 12.5
HER2: 19.3

In certain embodiments, further comprising determining if genomic DNA from the tumor sample comprises one or more clinically relevant mutations. In some embodiments, the clinically relevant mutation is a BRAF, KIT, NF, NRAS, or PTEN mutation.

In certain embodiments, it is determined if the one or more antibody-based therapies or one or more mutation-directed targeted therapies would more likely be effective.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Housekeeping Gene Selection

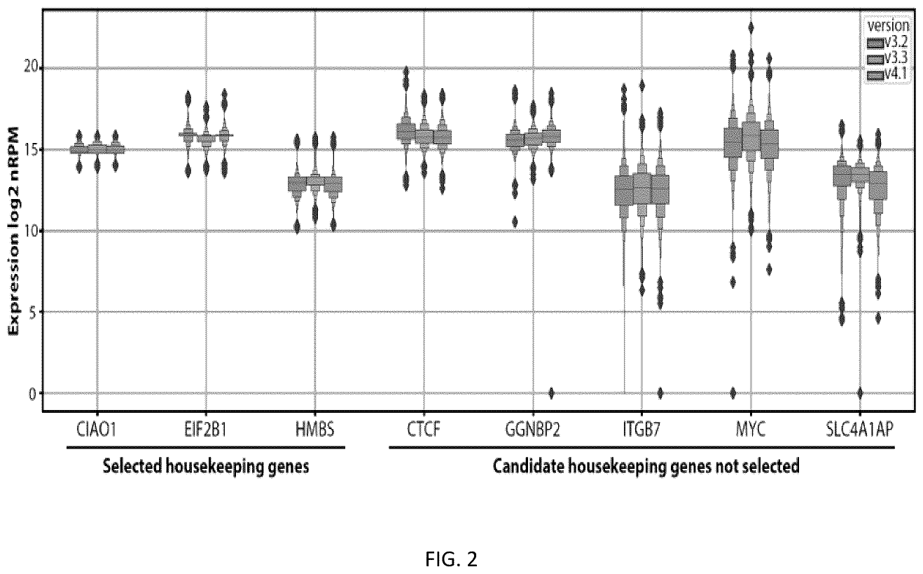

Selection of pan-cancer, pan-normal tissue, stable housekeeping genes is critical to the gene expression component of StrataEXP℠, given the desire to include both tumor (e.g., PD-L1) and non-tumor (e.g., PD-1) components and the need to be robust to variable tumor content across tissues. Candidate pan-cancer stable housekeeping genes were identified through a multi-step process. First, uniformly realigned, gene expression quantified, quantile normalized, and batch effect removed TCGA expression data (in fragments per kilobase per million [FPKM]) was downloaded for 6,875 tumor samples (from 18 tumor types).³⁷ Correlation of variation (CV) was determined for each gene per tumor type, and candidate housekeeping genes were ranked by the number of tumor types in which they ranked in the top 20 most stable genes (by lowest CV). Uniformly processed gene expression data (in transcripts per million [TPM]) from the 18 highest ranking housekeeping genes, 6 “positive control” genes included in the RNA component of the Oncomine Focus/Precision Assay (OPA), as well as the commonly used housekeeping gene GAPDH were then downloaded for 20,841 total samples contained in the MiPanda database³⁸, which includes 935 CCLE cancer cell lines (from 20 tumor types), 9,966 normal tissue samples (730 TCGA samples from 20 tissue types and 9,236 GTEX samples from 30 tissue types), and 9,940 TCGA cancer tissue samples (9,496 primary samples from 25 tumor types and 444 metastases from 16 tumor types). Given that most target genes are expressed at much lower levels than typical control genes (such as GAPDH), we prioritized inclusion of pan-cancer, pan-normal tissue stable genes with the lowest average expression (in TPM) as candidate housekeeping genes for the multiplex RNAseq component of the StrataEXP℠ component. Subsequently, a combination of 3 housekeeping genes (EIF2B1, CIAO1 and HMBS) were identified during clinical trials, that had the most stable expression patterns across tumor samples and the widest limit of quantification (LOQ) range (FIG. 2). All subsequent validated versions of the gene expression panel(s) use these three housekeeping genes.

To further ensure the selected housekeeping genes are suitable for use in normalizing expression in each individual sample, the method may further include one or both of modeling the expected distribution of housekeeping gene expression (Mahalanobis distance of sample housekeeping target expression <4 standard deviations away from the population) and confirming that each housekeeping gene is within its LOQ (equivalent to reportable range given this includes both upper and lower bounds). Additional quality control may be performed through inclusion of the same two FFPE RNA samples (GIAB/AshF and a custom PD-L1 high FFPE MSI-H cell line block with multiple engineered variants [HZ block]) in all clinical runs of the gene expression panel subjected to the same approach.

Unlike traditional RNAseq, where overall gene quantification values (e.g., in FPKM) are reported and are dependent on alignment approaches, our multiplex PCR based RNAseq approach enables unambiguous read assignment to each target gene amplicon. For quality control, we have included two separate amplicons targeting different exon-exon junctions of the same gene for several expression targets, including PDCD1, CD274 (PD-L1), and ADAM12. Highly correlated expression of these replicate amplicons can be used as additional quality control.

### StrataEXP℠

StrataEXP℠ is a laboratory-developed quantitative multi-gene RNA expression profiling test that leverages targeted RNA multiplex PCR-based sequencing to assess quantitative expression of >100 target genes. At this time, there are 8 clinically reported biomarkers that can be targeted by FDA-approved or investigational therapies, such as antibodies, antibody drug conjugates, radiopharmaceuticals, and immunotherapies, plus a prognostic quantitative proliferation score, that is listed on the StrataEXP℠ report. The reported clinical biomarkers from StrataEXP℠ are HER2, PD-L1, AR (androgen receptor), Nectin-4, TROP2, NaPi2b, FOLR1 (folate receptor), PSMA, and a multigene proliferation index (averaged expression of UBE2C and TOP2A expression).

Collection of data has been ongoing since original validation of StrataEXP℠ and now includes dozens of high-quality independent clinical library replicates and over 100 orthogonal PD-L1 IHC, HER2, Ki67, TROP2 and Nectin-4 test results. In-silico methods were used to leverage the data to refine the assay's per-transcript limit of quantification via clinical library replicates and clinical reporting threshold for PD-L1, HER2, and proliferation index via orthogonal test results.

### Materials and Methods

### Transcript Limit of Quantification

We determined LOQ and linearity for individual target genes by determining the lowest nRPM that can be precisely quantified in replicate RNA aliquots subjected to repeat multiplex PCR based library preparation (followed by templating and sequencing).

Importantly, independent library preparations represent independent RNA aliquots, cDNA (from reverse transcription) and library preparation, so the nRPM level below which repeat samples show increased dispersion represents the lowest amount of RNA library that can be precisely quantified.

Therefore, using a cohort of >100 clinical FFPE tumor samples submitted for StrataNGS® testing and subjected to repeat testing from the same isolated RNA, we determined the LOQ for all individual target gene amplicons by evaluating the weighted root mean square error (WRMSE) using a 40 sample window (beginning with the highest expressing target gene expression in replicate #1) and evaluated the minimum WRWSE, as well as additional windows (again beginning with the highest expressing target gene expression in replicate #1) where the WRMSE is first observed to be below the 50th, 25th, 15th and 10th quantile. Using this approach, the clinical implications of residual error distribution were weighed vs. the overall linearity and dynamic range of quantification in setting the most appropriate LOQ for each target gene amplicon. Linearity was thus determined by the concordance correlation coefficient for each target gene after setting all sub-LOQ values to LOQ. No upper LOQ is established as there is essentially no chance of clinical misinterpretation of a value higher than that established in this approach given the observed linearity. Hence, the reportable range for each amplicon is floored at the LOQ but has not upper limit. In one embodiment, LOQ values are those found in Table 1 below.

TABLE 1
Per-amplicon limits of quantification

| Amplicon | LOQ | Amplicon | LOQ | Amplicon | LOQ |
|---|---|---|---|---|---|
| ADAM12.E10E11.NM_003474.4 | 9.22 | FOXP3.E6E7.NM_001114377.1 | 10.41 | NTRK2.3p.E18E19 | 9.01 |
| ALK.3p.E22E23 | N/A | GPNMB.E4E5.NM_001005340.1 | 17.20 | NTRK2.5p.E4E5 | 10.40 |
| ALK.5p.E14E15 | 10.40 | HAVCR2.E2E3.NM_032782.3 | 14.21 | NTRK3.3p.E17E18 | 11.82 |
| ALK.Int19.ATI | N/A | IGF1R.E14E15.NM_000875.3 | 13.20 | NTRK3.5p.E4E5 | 9.53 |
| ALPPL2.E1E2.NM_031313.2 | 4.70 | LAG3.E7E8.NM_002286.5 | 12.10 | PDCD1.E4E5.NM_005018.2 | 7.13 |
| AR-AR.A3A4.V7 | 6.77 | MET.E15E16 | 14.12 | PVRL4.E3E4.NM_030916.2 | 8.78 |
| AR.E7E8 | 11.23 | MGMT.E3E4.NM_002412.3 | 14.71 | RET.3p.E18E19 | 12.15 |
| AXL.E13E14.NM_001699.4 | 12.82 | NM_000616:CD4 | 12.27 | RET.5p.E2E3 | 6.56 |
| CA6.E3E4.NM_001215.2 | N/A | NM_000619:IFNG | N/A | ROR2.E5E6.NM_004560.3 | 8.57 |
| CD274.E4E5.NM_014143.3 | 10.05 | NM_001067:TOP2A | 11.75 | ROS1.3p_NM_002944.2.e38e39 | 10.02 |
| CTLA4.E1E2.NM_001037631.2 | 10.92 | NM_002164:IDO1 | 8.90 | ROS1.5p_NM_002944.2.e11e12 | 11.31 |
| DLL3.E5E6.NM_016941.3 | 10.25 | NM_005018:PDCD1 | 11.92 | SLC34A2.E7E8.NM_001177998.1 | 10.97 |
| EGFR.E26E27 | 11.24 | NM_006144:GZMA | 8.09 | SLC39A6.E4E5.NM_001099406.1 | 16.13 |
| EGFR.E6E7 | 14.19 | NM_007019:UBE2C | 11.90 | TACSTD2.E1.NM_002353.2 | 10.02 |
| ENPP3.E22E23.NM_005021.3 | 13.20 | NM_014143:CD274 | 11.88 | TCF7.E3E4.NM_001134851.2 | 14.67 |
| ERBB2.E14E15.NM_001005862.1 | 11.49 | NM_025239:PDCD1LG2 | 15.30 | TIGIT.E2E3.NM_173799.3 | 5.43 |
| ERBB2.E6E7.NM_001005862.1 | 13.26 | NM_171827:CD8A | 12.76 | TNFRSF4.E2E3.NM_003327.3 | 11.80 |
| ERBB3.E26E27.NM_001982.3 | 12.56 | NTRK1.3p.E14E15 | 13.58 | TNFRSF9.E2E3.NM_001561.5 | 9.62 |
| FOLH1.E13E14.NM_001014986.1 | 12.88 | NTRK1.5p.E5E6 | 12.73 | VTCN1.E5E6.NM_024626.2 | 10.45 |

LOQ: limit of quantification.
N/A: amplicons whose limit of quantification could not be determined and will not be included in clinical reporting.
*: a correlation threshold of 0.6 was used for SLC39A6 (vs. 0.7 for all others) due to limited dynamic range in the replicate set.

### Histology

All FFPE blocks used in this method underwent sectioning to produce a single slide for hematoxylin and eosin (H&E) staining for pathology evaluation. A single H&E slide from the unstained FFPE slide set was used in the validation. The slides were reviewed by a board-certified anatomic pathologist to confirm the presence of tumor content, estimate tissue surface area and tumor content (TC; percentage of tumor nuclei/total nuclei), and to determine the number of punches, curls, or slides required to be cut or used for tissue isolation. The H&E slide was marked by the reviewing pathologist as needing macrodissection to enrich for tumor from noninvolved surrounding tissue, and up to 5×10 μm sections (from FFPE blocks) or 9×5 μm sections (from unstained slides) are used for nucleic acid isolation for clinical samples.

### DNA and RNA Sequencing

Samples passing pathology review were batched for nucleic acid extraction using VERSANT Sample Preparation 1.0 Reagents (Siemens, Malvern, Pennsylvania) on automated liquid handlers. One aliquot of isolated nucleic acid was treated with DNase I digested to provide an RNA-only sample. Quantification of purified nucleic acid was performed using the Quant-iT dsDNA High Sensitivity Assay Kit (ThermoFisher Scientific, Waltham, Massachusetts) or the Quant-iT RNA Assay Kit (ThermoFisher Scientific, Waltham, Massachusetts) on automated liquid handlers and Tecan Infinite 200 PRO fluorescent plate readers (Tecan Group Ltd., Morrisville, North Carolina). Normalization was performed on automated liquid handlers to 16 ng input DNA (per panel) and 15 ng input RNA (replicate reactions of the panel are run). RNA samples were reverse transcribed to cDNA using the Ion AmpliSeq Library Kit 2.0 (ThermoFisher Scientific, Waltham, Massachusetts), and SuperScript IV VILO Master Mix (ThermoFisher Scientific, Waltham, Massachusetts) on automated liquid handlers. DNA and RNA (cDNA) library preparation was completed using Agencourt AMPure beads (Beckman Coulter Life Sciences, Indianapolis, Indiana), the Ion AmpliSeq Library Kit 2.0, and the Ion Library Equalizer Kit (ThermoFisher Scientific, Waltham, Massachusetts). StrataEXP℠ utilizes the Ion S5XL/Prime sequencing workflow (ThermoFisher Scientific, Waltham, Massachusetts) and Ampliseq library preparation workflow for both the DNA and RNA components. Up to 16 clinical tumor samples can be sequenced on a single Ion 550 chip. The Ion 550 chip and kit-chef, along with Oncomine TML assay, and the Strata RNA panel were the primary components used as part of the process.

Reproducibility within and between operators was established using separate replicate RNA samples isolated from FFPE tumor samples. Twenty-seven samples were assessed by two operators a total of two times: operator 1 assessed the samples two times on two different dates and operator 2 assessed the samples two times on different days. Each operator performed templating and sequencing sequentially on the different Ion Chefs and alternate runs on different S5XL sequencing instruments for each run. For each sample, the minimum and maximum nRPM for each target gene across all replicates was plotted.

### Quantitative Reverse Transcription Polymerase Chain Reaction

Individual non-chimeric transcript target quantification from the RNA multiplex PCR-based panel used for the gene expression component of StrataEXP℠ was compared to orthogonal quantification by hydrolysis probe based quantitative reverse transcription polymerase chain reaction (qRT-PCR) gene expression. Clinical FFPE tumor samples from StrataNGS® testing and 3 control RNA samples were subjected to the multiplex RNAseq component of StrataEXP℠ and qRT-PCR on replicate RNA aliquots. Samples were serially diluted prior to qPCR processing. Two to 20 ng RNA underwent reverse transcription using Invitrogen SuperScript IV VILO Master Mix (ThermoFisher Scientific, Waltham, Massachusetts) and pre-amplification using Applied Biosystems TaqMan PreAmp Master Mix (ThermoFisher Scientific, Waltham, Massachusetts) using a pool of 48 individual Taqman primer/hydrolysis probe assays and 14 cycles. Quantitative PCR was then performed in duplicate on a Quantstudio 3 Real Time PCR system using a 1:20 dilution of amplified product per qPCR reaction and Applied Biosystems TaqMan Fast Universal PCR Master Mix (ThermoFisher Scientific, Waltham, Massachuse). Individual amplicon level thresholds and baselines were set during the exponential amplification phase to determine cycle crossing threshold (Ct) values. Samples with duplicate qPCR values >2 Ct difference were excluded unless both values were >30 or singlicate experiments were performed. All undefined Ct values were considered as having a Ct of 40. For StrataNGS®, target gene expression was determined as normalized target gene expression (nRPM34), using a common normal FFPE reference sample run in all clinical and validation runs as the reference sample, and the specific housekeeping genes for that multiplex RNAseq panel and/or version.

### Data Analysis

Sequencing data was processed using versioned, end-to-end validated bioinformatic pipelines based on Torrent Suite version 5.8, Ion Reporter version 5.2 (ThermoFisher Scientific, Waltham, Massachusetts), Variant Effect Predictor version 95.3 (Ensembl; EMBL-EBI, Hinxton, Cambridge, UK), and Strata Bioinformatics Pipeline version 4.0 (Strata Oncology, Ann Arbor, Michigan). The Torrent Suite pipeline performed sequence demultiplexing; the Ion Reporter pipeline performed RNA read mapping; the Strata Bioinforatics pipeline performed read count normalization and quality control analysis.

### Methodology for Obtaining Orthogonal HER2, PD-L1, and Ki67 Information.

Optical character recognition and natural language processing were used to prioritize accompanying pathology reports received with StrataNGS® test requests for abstraction of IHC biomarker results by trained reviewers according to a documented SOP into a clinical database. Where orthogonal IHC biomarkers and StrataNGS® were performed on different specimens from the same case (pathology accession) that are presumed to come from the same tumor (e.g. single case with a primary colon cancer, lymph node metastasis, liver metastasis resection), testing was considered as performed on the same specimen and suitable for comparison (representing usual clinical practice). Where orthogonal biomarker and StrataNGS® testing were performed on different specimen from the same case (pathology accession) where grossly or histologically distinct tumors were described and commented on in the specimen report, testing was considered as performed on distinct specimens and not suitable for comparison. For breast cancer biomarkers, cases were excluded if they only referenced results from a previous sample, and only cases with quantitative results (with reference to CAP/ASCO guidelines) were included). Additionally, for Ki67, orthogonal results were excluded if a range of IHC expression >20% was provided (e.g. Ki67 staining reported as “>50%” was excluded); for orthogonal IHC results with <=20% range, the average of the range was used (e.g. Ki67>80%=90%). HER2 IHC expression was binned into the three currently distinct clinical groups (negative [0 or 1+], 2+, and 3+).

StrataEXP℠ Highly Concordant with qRT-PCR.

In order to validate the quantitative RNA expression data output obtained by StrataEXP℠, 26 tissue samples were prepared and subjected to StrataEXP℠ and qRT-PCR to quantify target RNA transcripts as outlined above. Expression levels of 36 target genes as determined by StrataEXP℠ and qRT-PCR were plotted and a correlation between them calculated. With a coefficient of determination of r²=0.749, it was determined that StrataEXP℠ is highly concordant with qRT-PCR, thus validating StrataEXP℠ as a viable and accurate method for quantifying the expression of select target genes.

### Nectin-4 Threshold

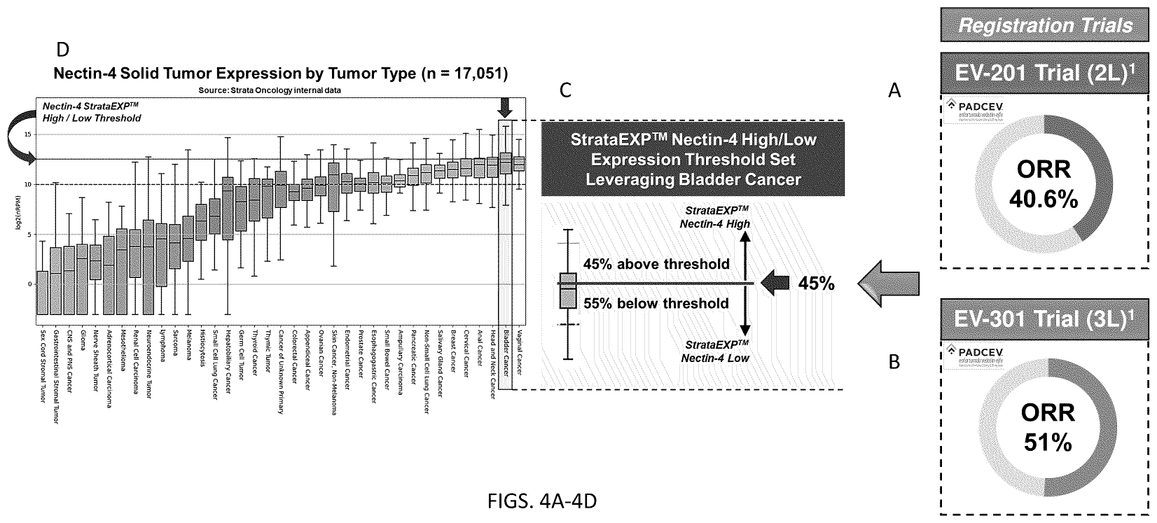

Nectin-4, an immunoglobulin-like transmembrane protein, is highly expressed in many different tumor types. Using the techniques and protocols discussed above, comprehensive genomic and transcriptomic profiling of advanced solid tumors within the Strata Trial cohort was performed (n=17,051). As shown in FIG. 4D, Nectin-4 is expressed in many different tumor types, yet Nectin-4 had the highest mean expression in bladder cancer.

FIGS. 4A-4B demonstrate that the objective response rate to anti-Nectin-4 therapy enfortumab vedotin in a recent clinical trial in second line and third line bladder cancer patients is 51% and 40.6%, respectively. As median Nectrin-4 expression is highest in bladder cancer patients and as target expression level is associated with response to antibody/antibody drug conjugates targeting the overexpression, the average objective response rate of 45% was used to set the StrataEXP℠ threshold for high/low Nectrin-4 of approximately 12.5 as shown in FIGS. 4C-4D. Essentially, this identifies cancer patients likely to respond to anti-Nectin-4 therapy as those with a Nectrin-4 expression at or above bladder cancer patients with the top 45% of Nectin-4 overexpression. Notably, while bladder cancer has the highest proportion of tumors >12.5, many other tumor types had sub-populations with similar over-expression. Apart from urothelial carcinomas, Nectin-4 was most frequently over-expressed in squamous cell carcinomas, including cervical, head & neck, vulvar, esophagogastric, cutaneous, anal, and lung.

### TROP2 Threshold

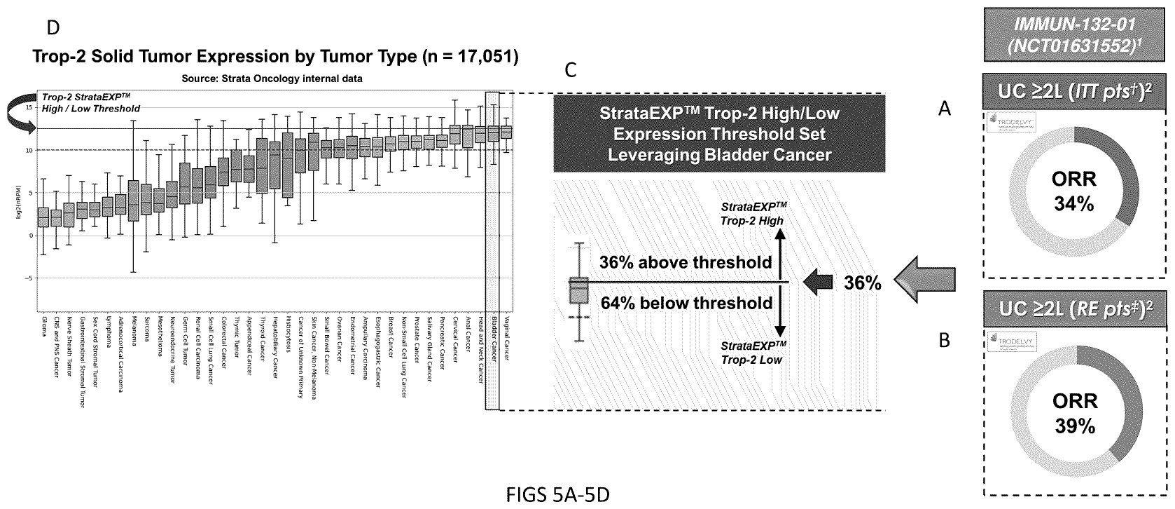

TROP2 is type-I transmembrane glycoprotein highly expressed in various types of adenocarcinomas and its expression has been correlated with tumor aggressiveness. Using the techniques and protocols discussed above, comprehensive genomic and transcriptomic profiling of advanced solid tumors within the Strata Trial cohort was performed (n=17,051). As demonstrated by FIG. 5D, many different cancers have high expression of TROP2. Of particular note are vaginal cancer, bladder cancer, head and neck cancer, anal cancer, cervical cancer and pancreatic cancer, which all show similar TROP2 overexpression. A significant number of additional cancer groups also demonstrate subgroups which contain similar TROP2 overexpression as the aforementioned cancers groups.

As shown in FIGS. 5A-5B, in a recent clinical study that administered the anti-TROP2 therapeutic Sacituzumab govitecan-hziy to second line or higher bladder cancer patients, the objective response rate was 34% and 39% based upon assignment to the intention-to-treat or response evaluable patient cohort, respectively. As was likewise done when setting a threshold for Nectin-4 expression levels, the StrataEXP℠ TROP2 high/low expression threshold was set based upon the approximate average objective response rate of the two bladder cancer patient cohorts and had a value of approximately 12.5 log 2 (nRPM). This measure similarly indicates as likely to respond to anti-TROP2 therapy, who have cancer demonstrating overexpression of TROP2 at or above the level of bladder cancer patients with highest 36% of TROP2 overexpression. As noted previously, many other tumor types had sub-populations with similar over-expression as those in bladder cancer and thus application of the method includes such a subset of cancers.

### HER2 Reporting Threshold

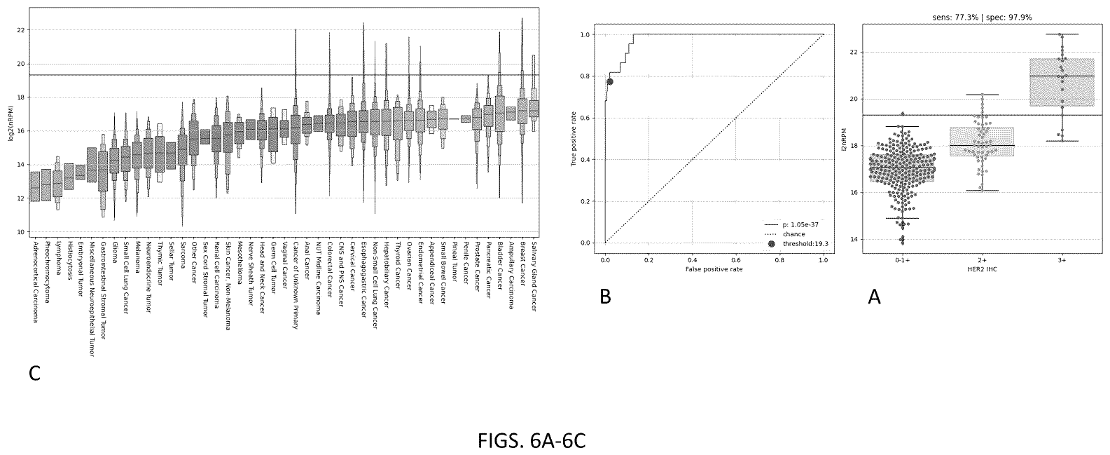

For HER2, given the routine clinical use of IHC to assess expression in breast cancer, we identified StrataNGS®/StrataEXP℠ tested patients with clinical HER2 IHC in their accompanying pathology reports. We identified a cohort of 312 breast cancer samples with orthogonal HER2 IHC and StrataEXP℠ testing. We defined a StrataEXP℠ HER2 cutoff of approximately 19.3 to define the population most likely to respond to HER2 directed therapy based on high IHC expression, which yielded a 77.3% sensitivity and a 97.9% specificity (FIG. 6A). This threshold is then applied to all StrataEXP℠ tested samples (n=23,514) to generate a pan-cancer threshold of high HER2 expression by StrataEXP℠ (FIG. 6B).

### PD-L1 Reporting Threshold

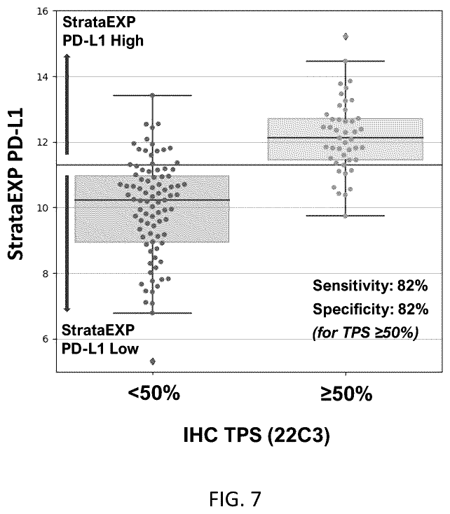

The PD-L1 reporting threshold was determined via setting the threshold to maximize utility in a cohort of 136 NSCLC samples with orthogonal IHC testing results (Clone 22C3; Dako, Carpenteria, CA). A log 2 (nRPM) threshold of approximately 11.3 yielded 82.2% sensitivity and 82.4% specificity vs IHC-based TPS ≥50% status (amplicon: NM_014143: CD274). Individual data points are shown in FIG. 7.

### Proliferation Index (Ki-67) Reporting

For proliferation index, the average of UBE2C+TOP2A expression was compared by manual analysis to Ki-67 index (proliferation index expression reporting) by IHC in a cohort of 310 clinical samples, and the Pearson's correlation coefficient=0.823.

The proliferation index reportable range was established via the lowest average scoring tumor type to the highest average scoring tumor type in a population analysis of 18,352 clinical samples. The per-tumor type averages indicate the reportable range is 7.79 to 12.1 log 2 (nRPM), defined by the tumor type with the minimum average score (Gastrointestinal Stromal Tumor) and the highest (Small Cell Lung Cancer) (Table 2).

TABLE 2
Average proliferation scores per tumor type

| Tumor Type | n | Average proliferation index |
|---|---|---|
| Adrenocortical Carcinoma | 15 | 15.28 |
| Ampullary Carcinoma | 27 | 15.59 |
| Anal Cancer | 56 | 16.63 |
| Appendiceal Cancer | 31 | 15.01 |
| Bladder Cancer | 435 | 16.23 |
| Breast Cancer | 1537 | 15.40 |
| CNS and PNS Cancer | 88 | 13.94 |
| Cancer of Unknown Primary | 919 | 15.63 |
| Cervical Cancer | 107 | 16.58 |
| Colorectal Cancer | 1774 | 16.23 |
| Endometrial Cancer | 607 | 16.00 |
| Esophagogastric Cancer | 656 | 16.23 |
| Gastrointestinal Stromal Tumor | 87 | 13.44 |
| Germ Cell Tumor | 24 | 16.24 |
| Glioma | 700 | 14.51 |
| Head and Neck Cancer | 363 | 16.25 |
| Hepatobiliary Cancer | 288 | 14.95 |
| Lymphoma | 72 | 15.76 |
| Melanoma | 457 | 15.23 |
| Mesothelioma | 56 | 14.85 |
| Nerve Sheath Tumor | 12 | 16.07 |
| Neuroendocrine Tumor | 205 | 14.72 |
| Non-Small Cell Lung Cancer | 2180 | 15.45 |
| Ovarian Cancer | 845 | 15.48 |
| Pancreatic Cancer | 593 | 15.33 |
| Prostate Cancer | 818 | 14.15 |
| Renal Cell Carcinoma | 287 | 14.33 |
| Salivary Gland Cancer | 84 | 14.91 |
| Sarcoma | 469 | 15.63 |
| Sex Cord Stromal Tumor | 19 | 14.60 |
| Skin Cancer, Non-Melanoma | 83 | 16.05 |
| Small Bowel Cancer | 58 | 16.01 |
| Small Cell Lung Cancer | 196 | 17.64 |
| Thymic Tumor | 24 | 15.34 |
| Thyroid Cancer | 146 | 13.79 |
| Vaginal Cancer | 40 | 16.06 |

### REFERENCES

1 Imielinski, M., Guo, G. & Meyerson, M. Insertions and Deletions Target Lineage-Defining Genes in Human Cancers. Cell 168, 460-472 e414, doi: 10.1016/j.cell.2016.12.025 (2017).
2 Zhu, Y. et al. High-throughput proteomic analysis of FFPE tissue samples facilitates tumor stratification. Mol Oncol 13, 2305-2328, doi: 10.1002/1878-0261.12570 (2019).
3 Bhagwat, A. S. & Vakoc. C. R. Targeting Transcription Factors in Cancer. Trends Cancer 1, 53-65, doi: 10.1016/j.trecan.2015.07.001 (2015).
4 Tanney, A. & Kennedy, R. D. Developing mRNA-based biomarkers from formalin-fixed paraffin-embedded tissue. Per Med 7, 205-211, doi:10.2217/pme.10.8 (2010).
5 Volinsky, N. & Kholodenko, B. N. Complexity of receptor tyrosine kinase signal processing. Cold Spring Harb Perspect Biol 5, a009043, doi: 10.1101/cshperspect.a009043 (2013).
6 Kwak. E. L. et al. Anaplastic lymphoma kinase inhibition in non-small-cell lung cancer. N Engl J Med 363, 1693-1703, doi: 10.1056/NEJMoa1006448 (2010).
7 Garon, E. B. et al. Pembrolizumab for the treatment of non-small-cell lung cancer. N Engl J Med 372, 2018-2028, doi: 10.1056/NEJMoa1501824 (2015).
8 Dolled-Filhart, M. et al. Development of a Prototype Immunohistochemistry Assay to Measure Programmed Death Ligand-1 Expression in Tumor Tissue. Arch Pathol Lab Med 140, 1259-1266, doi: 10.5858/arpa.2015-0544-OA (2016).
9 Dolled-Filhart, M. et al. Development of a Companion Diagnostic for Pembrolizumab in Non-Small Cell Lung Cancer Using Immunohistochemistry for Programmed Death Ligand-1. Arch Pathol Lab Med 140, 1243-1249, doi: 10.5858/arpa.2015-0542-OA (2016).
10 Herbst, R. S. et al. Atezolizumab for First-Line Treatment of PD-L1-Selected Patients with NSCLC. N Engl J Med 383, 1328-1339, doi: 10.1056/NEJMoa1917346 (2020).
11 Doroshow, D. B. et al. PD-L1 as a biomarker of response to immune-checkpoint inhibitors. Nat Rev Clin Oncol 18, 345-362, doi: 10.1038/s41571-021-00473-5 (2021).
12 Gavrielatou, N., Shafi, S., Gaule, P. & Rimm, D. L. PD-L1 Expression Scoring: Non-Interchangeable, Non-Interpretable, Neither, or Both. J Natl Cancer Inst, doi: 10.1093/jnci/djab109 (2021).
13 Chan, T. A., Wolchok, J. D. & Snyder, A. Genetic Basis for Clinical Response to CTLA-4 Blockade in Melanoma. N Engl J Med 373, 1984. doi: 10.1056/NEJMc1508163 (2015).
14 Van Allen, E. M. et al. Genomic correlates of response to CTLA-4 blockade in metastatic melanoma. Science 350, 207-211, doi: 10.1126/science.aad0095 (2015).
15 Rizvi, N. A. et al. Cancer immunology. Mutational landscape determines sensitivity to PD-1 blockade in non-small cell lung cancer. Science 348, 124-128, doi: 10.1126/science.aaa1348 (2015).
16 Snyder, A. et al. Genetic basis for clinical response to CTLA-4 blockade in melanoma. N Engl J Med 371, 2189-2199, doi: 10.1056/NEJMoa1406498 (2014).
17 Hugo, W. et al. Genomic and Transcriptomic Features of Response to Anti-PD-1 Therapy in Metastatic Melanoma. Cell 165, 35-44, doi:10.1016/j.cell.2016.02.065 (2016).
18 Rosenberg, J. E. et al. Atezolizumab in patients with locally advanced and metastatic urothelial carcinoma who have progressed following treatment with platinum-based chemotherapy: a single-arm, multicentre, phase 2 trial. Lancet 387, 1909-1920, doi: 10.1016/S0140-6736(16)00561-4 (2016).
19 Le, D. T. et al. Mismatch repair deficiency predicts response of solid tumors to PD-1 blockade. Science 357, 409-413, doi: 10.1126/science.aan6733 (2017).
20 Marcus, L. et al. FDA Approval Summary: Pembrolizumab for the Treatment of Tumor Mutational Burden-High Solid Tumors. Clin Cancer Res 27, 4685-4689, doi: 10.1158/1078-0432.CCR-21-0327 (2021).
21 Modi, S. et al. Trastuzumab Deruxtecan in Previously Treated HER2-Low Advanced Breast Cancer. N Engl J Med, doi: 10.1056/NEJMoa2203690 (2022).
22 Chou, J. et al. TROP2 Expression Across Molecular Subtypes of Urothelial Carcinoma and Enfortumab Vedotin-resistant Cells. Eur Urol Oncol, doi:10.1016/j.euo.2021.11.005 (2022).
23 Coates, J. T. et al. Parallel Genomic Alterations of Antigen and Payload Targets Mediate Polyclonal Acquired Clinical Resistance to Sacituzumab Govitecan in Triple-Negative Breast Cancer. Cancer Discov 11, 2436-2445, doi: 10.1158/2159-8290.CD-21-0702 (2021).
24 Chu, C. E. et al. Heterogeneity in NECTIN4 Expression Across Molecular Subtypes of Urothelial Cancer Mediates Sensitivity to Enfortumab Vedotin. Clin Cancer Res 27, 5123-5130, doi: 10.1158/1078-0432.CCR-20-4175 (2021).
25 Gibney, G. T., Weiner, L. M. & Atkins, M. B. Predictive biomarkers for checkpoint inhibitor-based immunotherapy. Lancet Oncol 17, e542-e551, doi:10.1016/S1470-2045(16)30406-5 (2016).
26 Cristescu, R. et al. Pan-tumor genomic biomarkers for PD-1 checkpoint blockade-based immunotherapy. Science 362, doi: 10.1126/science.aar3593 (2018).
27 Fumet, J. D. et al. Prognostic and predictive role of CD8 and PD-L1 determination in lung tumor tissue of patients under anti-PD-1 therapy. Br J Cancer 119, 950-960, doi: 10.1038/s41416-018-0220-9 (2018).
28 Park, Y. J., Kuen, D. S. & Chung, Y. Future prospects of immune checkpoint blockade in cancer: from response prediction to overcoming resistance. Exp Mol Med 50, 1-13, doi: 10.1038/s12276-018-0130-1 (2018).
29 Ayers, M. et al. IFN-gamma-related mRNA profile predicts clinical response to PD-1 blockade. J Clin Invest 127, 2930-2940, doi: 10.1172/JCI91190 (2017).
30 Litchfield, K. et al. Meta-analysis of tumor- and T cell-intrinsic mechanisms of sensitization to checkpoint inhibition. Cell 184, 596-614 e514, doi: 10.1016/j.cell.2021.01.002 (2021).
31 Shohdy, K. S. et al. Functional comparison of exome capture-based methods for transcriptomic profiling of formalin-fixed paraffin-embedded tumors. NPJ Genom Med 6, 66, doi: 10.1038/s41525-021-00231-7 (2021).
32 Tomlins, S. A. et al. Development and Validation of StrataNGS, a Multiplex PCR, Semiconductor Sequencing-Based Comprehensive Genomic Profiling Test. J Mol Diagn 23, 1515-1533, doi: 10.1016/j.jmoldx.2021.08.005 (2021).
33 Tomlins, S. A. et al. Real-World Performance of a Comprehensive Genomic Profiling Test Optimized for Small Tumor Samples. JCO Precis Oncol 5, doi:10.1200/PO.20.00472 (2021).
34 Conroy J M, Pabla S, Glenn S T, et al. Analytical Validation of a Next-Generation Sequencing Assay to Monitor Immune Responses in Solid Tumors. J Mol Diagn. 2018; 20(1):95-109

What is claimed is:

1. A method of determining if one or more therapies are likely to be effective for treating a subject, comprising:
a. measuring the expression level of one or more gene products associated with the one or more therapies from a biological sample obtained from the subject, wherein the sample comprises nucleic acids derived from cells targeted by the one or more therapies;
b. measuring the expression level of at least one housekeeping gene selected from CIAO1, EIF2B1, and HMBS in the biological sample;
c. normalizing the expression levels of the one or more gene products to the at least one housekeeping gene to obtain normalized expression levels of the one or more gene products; and
d. determining that the one or more therapies are likely to be effective for treating the subject if the normalized expression levels of the one or more gene products are above a threshold level,
wherein the therapy comprises an antibody, bispecific antibody, antibody-drug conjugate, antibody fragment, radiopharmaceutical, Car-T cell, or engineered T cell receptor.

2. The method of claim 1, wherein the one or more therapies comprise two or more therapies.

3. The method of claim 1, wherein the one or more therapies comprise three or more therapies.

4. The method of claims 1-3, wherein the threshold level is the ranked expression level of the one or more gene products in a group of cells targeted by the one or more therapies corresponding to the percent of subjects not responsive to the one or more therapies.

5. The method of claims 1-4, wherein the biological sample is a tumor sample.

6. The method of claims 1-5, wherein the one or more therapies comprise an anti-Her2 antibody and the one or more gene products comprise Her2.

7. The method of claims 1-6, wherein the one or more therapies comprise an anti-Nectin-4 antibody and the one or more gene products comprise Nectin-4.

8. The method of claims 1-7, wherein the one or more therapies comprise an anti-TROP2 antibody and the one or more gene products comprise TROP2.

9. The method of claims 1-8, wherein it is determined that at least two therapies are likely to be effective and wherein the therapy more likely to be effective is identified based on having the highest relative expression of the associated gene product over the associated threshold level.

10. The method of claims 1-9, further comprising determining if genomic DNA from the biological sample comprises one or more clinically relevant mutations.

11. The method of claim 10, wherein it is determined if the one or more therapies or one or more mutation-directed targeted therapies would more likely be effective.

12. The method of claims 1-11, further comprising treating the subject with the one or more therapies or mutation-directed targeted therapy identified as likely to be effective.

13. A method of determining if one or more antibody-based therapies selected from anti-Her2, anti-Nectin-4, and anti-TROP2 antibody-based therapy is likely to be effective for treating a tumor in a subject, comprising:
a. measuring the expression level of Her2, Nectin-4, and TROP2 in a tumor sample obtained from the subject;
b. measuring the expression level of at least one housekeeping gene selected from CIAO1, EIF2B1, and HMBS in the tumor sample;
c. normalizing the expression levels of Her2, Nectin-4, and TROP2 to the at least one housekeeping gene to obtain normalized expression levels of the one or more gene products; and
d. determining that anti-Her2, anti-Nectin-4, or anti-TROP2 antibody-based therapy is likely to be effective if the normalized expression levels of Her2, Nectin-4, or TROP2 are above a threshold level.

14. The method of claim 13, further comprising determining which antibody-based therapy is more likely to be effective based on having the highest relative expression of the associated gene product over the associated threshold level.

15. The method of claims 13-14, further comprising determining if genomic DNA from the tumor sample comprises one or more clinically relevant mutations.

16. The method of claim 15, wherein it is determined if the one or more antibody-based therapies or one or more mutation-directed targeted therapies would more likely be effective.

17. A method of treatment of a subject with a therapy likely to be effective, comprising administering to said subject the therapy, wherein the therapy has been identified as likely to be effective by a method comprising:
a. measuring the expression level of a gene products associated with the therapy from a biological sample obtained from the subject, wherein the sample comprises nucleic acids derived from cells targeted by the therapy;
b. measuring the expression level of at least one housekeeping gene selected from CIAO1, EIF2B1, and HMBS in the biological sample;
c. normalizing the expression levels of the gene product to the at least one housekeeping gene to obtain a normalized expression level of the gene product; and
d. determining that the therapy is likely to be effective for treating the subject if the normalized expression level of the gene product is above a threshold level,
wherein the therapy comprises an antibody, bispecific antibody, antibody-drug conjugate, antibody fragment, radiopharmaceutical, Car-T cell, or engineered T cell receptor.

18. The method of claim 17, wherein a plurality of therapies have been identified as likely to be effective and it has been determined that one of the therapies is more likely to be effective based on having the highest relative expression of the associated gene product over the associated threshold level; and the therapy identified as more likely to be effective is administered to the subject.

19. A method of treatment of a subject with an antibody-based therapy identified as most likely to be effective selected from anti-Her2, anti-Nectin-4, and anti-TROP2 antibody-based therapy, comprising administering to said subject the antibody-based therapy identified as most likely to be effective, wherein the antibody-based therapy has been identified as most likely to be effective by a method comprising:
a. measuring the expression level of Her2, Nectin-4, and TROP2 in a tumor sample obtained from the subject;
b. measuring the expression level of at least one housekeeping gene selected from CIAO1, EIF2B1, and HMBS in the tumor sample;
c. normalizing the expression levels of Her2, Nectin-4, and TROP2 to the at least one housekeeping gene to obtain normalized expression levels of the one or more gene products;
d. determining that anti-Her2, anti-Nectin-4, or anti-TROP2 antibody-based therapy is likely to be effective if the normalized expression levels of Her2, Nectin-4, or TROP2 are above a threshold level; and
e. determining which antibody-based therapy is more likely to be effective based on having the highest relative expression of the associated gene product over the associated threshold level.
